(19) United States
(12) Patent Application Publication
(10) Pub. No.: US 2022/0214322 A1
(43) Pub. Date: Jul. 7, 2022

(54) AIR POLLUTANTS CONCENTRATION FORECASTING METHOD AND APPARATUS AND STORAGE MEDIUM

(71) Applicant: Tsinghua University, Beijing (CN)

(72) Inventors: Shiji SONG, Beijing (CN); Gao HUANG, Beijing (CN); Zhuofan XIA, Beijing (CN)

(73) Assignee: Tsinghua University, Beijing (CN)

(21) Appl. No.: 17/562,109

(22) Filed: Dec. 27, 2021

(30) Foreign Application Priority Data
Jan. 7, 2021 (CN) 202110017772.6

(51) Int. Cl. G01N 33/00; G06K 9/62; G06M 3/04

(52) U.S. Cl. CPC G01N 33/0034; G06M 3/04; G06K 9/6262; G06K 9/6256

(57) ABSTRACT

A method, apparatus and storage medium for forecasting air pollutant concentration, including: constructing a training set, a validation set and a test set based on a data set; the data set is obtained by collecting pollutant concentration data and meteorological data in a predetermined length of time in a target area; constructing an adjacent matrix A of a graph structure based on the spatial distribution of monitoring stations in the target area; establishing a neural network model F(x;Θ|A), where x is the input data of it, including pollutant concentration data and meteorological data within predetermined time period, training the neural network model using the data of the training set, adjusting the parameters Θ of the neural network model using the data of the validation set and the data of the test set, and obtaining the modified neural network model; using the modified neural network model for air pollutant concentration forecasting.

### CROSS REFERENCE OF RELATED APPLICATIONS

This application is based upon and claims priority to Chinese Patent Application No. 202110017772.6, filed on Jan. 7, 2021, the entire contents of which are incorporated herein by reference.

### TECHNICAL FIELD

The present disclosure relates to the technical field of computers, and particularly relates to an air pollutants concentration forecasting method and apparatus and a storage medium.

### BACKGROUND

The method of monitoring through air quality monitoring stations is the most commonly used method for air quality perception and air pollution observation method, which has the characterizes in high measurement accuracy and good stability. However, the spatial distribution of monitoring stations in China is too sparse, which makes it difficult to provide effective and accurate data for analysis and research. Therefore, in the view of the existing data acquisition status, in order to effectively analyze air pollution particulate matter, it is important to adopt a reasonable data analysis method.

In some technologies, data modeling methods for air pollutant concentrations mainly include theoretical-based methods and statistical-based methods, which can forecast current or future air pollutant concentrations at a certain time based on historical air pollutant concentration monitoring data. However, none of them can integrate and utilize the temporal and spatial dynamic characteristics of air pollutants, and the generalization ability and forecast accuracy are weak.

### SUMMARY

The following is an overview of the subject matter described in detail herein. This overview is not intended to limit the scope of protection of the claims.

This disclosure embodiment provides a method for forecasting air pollutant concentrations, which may improve generalization capability and prediction accuracy:

Embodiments of the present disclosure provide a method for forecasting air pollutant concentrations, comprising:

constructing a training set, a validation set and a test set from the data set;

aforementioned data set is obtained by collecting pollutant concentration data and meteorological data for a predetermined length of time in the target area;

constructing the adjacent matrix A of the graph structure based on the spatial distribution of monitoring stations in aforementioned target area;

building a neural network model F(x; η|A), x being input data for aforementioned neural network model, including aforementioned pollutant concentration data and meteorological data for a predetermined time period, training aforementioned neural network model using data from aforementioned training set, adjusting parameters Θ of aforementioned neural network model using data from aforementioned validation set and data from the test set, and obtaining a modified neural network model;

using the modified neural network model described to forecast air pollutant concentration.

Embodiments of the present disclosure also provide an air pollutant concentration forecasting device comprising a memory and a processor, where memory is for storing a program for performing air pollutant concentration forecasting; and processor is for reading the aforementioned program for performing air pollutant concentration forecasting and executing an air pollutant concentration forecasting method as described above.

Embodiments of the present disclosure also provide a computer-readable storage medium storing computer-executable instructions, described computer-executable instructions for performing an air pollutant concentration forecasting method as described above.

This disclosure embodiment uses a graph neural network-based air pollutant concentration forecasting method to integrate and utilize the temporal and spatial dynamic characteristics of air pollutants, which not only may capture the air pollutant concentration changes in a larger area more effectively, but also improves the efficiency and accuracy of forecasting, while having a high generalization capability for practical disclosure.

Other features and advantages of the present disclosure will be set forth in the subsequent specification and, in part, become apparent from the specification or are understood by implementing the present disclosure. Other advantages of the present disclosure may be realized and obtained by the embodiments described in the specification as well as in the accompanying drawings.

After reading and understanding the accompanying drawings and detailed description, other aspects may be understood.

### BRIEF DESCRIPTION OF THE DRAWINGS

The accompanying drawings are used to provide an understanding of the technical solutions of the present disclosure and form part of the specification, together with the embodiments of the present disclosure, for the purpose of explaining the technical solutions of the present disclosure and do not constitute a limitation of the technical solutions of the present disclosure.

FIG. 1 shows a flow chart of the method for forecasting air pollutant concentrations in an embodiment of the present disclosure;

FIG. 2 shows a schematic diagram of the neural network model in an embodiment of the present disclosure;

FIG. 3 shows a schematic diagram of the air pollutant concentration forecasting device in this disclosure embodiment; and

FIG. 4 shows a schematic diagram of an air pollutant concentration forecasting device in yet another realized form in this disclosure embodiment.

### DETAILED DESCRIPTION OF THE EMBODIMENTS

The present disclosure describes a plurality of embodiments, but the description is exemplary and not limiting, and it will be apparent to one of ordinary skill in the art that there may be many more embodiments and implementations within the scope of the embodiments described in the present disclosure. Although many possible combinations of features are illustrated in the accompanying drawings and discussed in the specific embodiments, many other combinations of the disclosed features are possible. Except where specifically limited, any feature or element of any embodiment may be used in combination with any other feature or element in any other embodiment, or may be substituted for any other feature or element in any other embodiment.

The present disclosure includes and contemplates combinations with features and elements known to those of ordinary skill in the art. The embodiments, features and elements already disclosed in this disclosure may also be combined with any conventional feature or element to form a unique inventive embodiment limited by the claims. Any feature or element of any embodiment may also be combined with a feature or element from another inventive embodiment to form another unique inventive embodiment as defined by the claims. Thus, it should be understood that any of the features illustrated and/or discussed in this disclosure may be implemented individually or in any suitable combination. Accordingly, embodiments are not limited other than by the limitations made in accordance with the appended claims and equivalent substitutions thereof. In addition, various modifications and changes may be made within the scope of protection of the appended claims.

In addition, in describing representative embodiments, the specification may have presented the method and/or process as a particular sequence of steps. However, to the extent that the method or process does not depend on a particular sequence of steps as described herein, the method or process should not be limited to the particular sequence of steps described. Other sequences of steps are possible, as will be understood by those of ordinary skill in the art. Thus, the particular order of steps set forth in the specification should not be construed as a limitation of the claims. Furthermore, the claims directed to the method and/or process should not be limited to steps that perform them in the order in which they are written, and it will be readily understood by those of ordinary skill in the art that these orders may be varied and still remain within the spirit and scope of the embodiments of the present disclosure.

As stated in the background technology, the data modeling methods for air pollutant concentrations in some technologies mainly include theoretical-based methods as well as statistical-based methods, and the inventors of this disclosure have keenly identified the following problems with these methods in practice.

Theory-based methods, also known as numerical forecasting, are one of the common techniques of modern weather forecasting. These methods are generally based on the theory of atmospheric dynamics and simulate the reactions of molecules of air pollutant particles in the atmosphere and their dispersion, and give simulations of air pollutant concentrations by means of numerical calculations. This type of method gives a better picture of the atmospheric motion and a better interpretation of meteorological phenomena. However, due to the extremely nonlinear and time-varying nature of meteorological system changes, modeling based on atmospheric dynamics theory is not effective in modeling the dynamics of rapid changes in a short period of time, and it is often necessary in practice to use the joint forecasting of multiple systems to enhance the accuracy and stability of forecasts.

Forecasting algorithms based on statistical methods, such as the autoregressive integrated moving average model (ARIMA), support vector machine regression (SVR) and deep neural networks, are able to be used in various scenarios. Since statistical-based methods discard complex mechanistic modeling, they are fast, simple and less computationally intensive, and are suitable for analyzing short-term time trends.

However, these methods mentioned above are not able to make integrated use of the temporal and spatial dynamic properties of air pollutants and do not have a strong generalization capability and the accuracy of forecasting is not high.

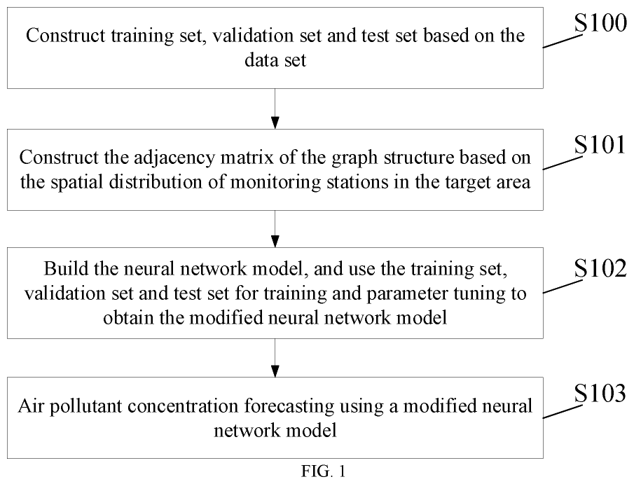

As shown in FIG. 1, embodiments of the present disclosure provide a method for forecasting air pollutant concentrations, comprising:

S100: constructing a training set, a validation set and a test set based on the data set; the data set is obtained by collecting pollutant concentration data and meteorological data in the target area for a predetermined length of time;

S101: construct the adjacent matrix A of the graph structure based on the spatial distribution of monitoring stations in the target area;

S102: establish a neural network model F(x; Θ|A), x is the input data of the neural network model, including pollutant concentration data and meteorological data in the predetermined time period, train the neural network model using the data of the training set, adjust the parameters Θ of the neural network model using the data of the validation set and the data of the test set, and obtain the modified neural network model;

S103: Air pollutant concentration forecasting using the modified neural network model.

When performing step S100, any target area may be selected according to demand, such as selection by province, city, county, etc., or only a certain area may be selected, not limited to a certain administrative boundary, which is not limited by this disclosure embodiment.

After the target area is selected, pollutant concentration data and meteorological data of the target area are collected for a predetermined period of time, and the “predetermined period of time” may be set according to actual needs, such as a period of time of more than 3 months, and the time resolution of the data may be set according to needs, such as 1 hour. The time resolution of the data may be set as needed, such as 1 hour. Air pollutant concentration data may include PM10, PM2.5, O3, SO2, NOx, etc., and meteorological data may include temperature, humidity, wind speed, wind direction, atmospheric pressure, etc. The collected pollutant concentration data and meteorological data for a predetermined length of time may be calculated using a linear interpolation model to supplement the missing values therein, i.e., the linear average of multiple data points in the time period before and after the missing part is used to fill in the values of the temporally missing data points, and the data set is obtained after collation. After obtaining the dataset, the dataset may be used to construct the training set, validation set and test set to train and tune the neural network model.

The spatial distribution of the monitoring stations in the target area in step S101 refers to the spatial distribution of the air quality monitoring stations arranged in the target area.

After determining the target region, either the data set may be collected and organized first, or the adjacent matrix A of the graph structure may be constructed first, and the order of execution of these two steps may be chosen as needed.

In step S102, the input data x of the neural network model F(x; Θ|A) may include pollutant concentration data and meteorological data within a predetermined time period, and the user may set the length of the “predetermined time period” according to the need, and the neural network model may predict the pollutant concentration data in the future period (the length of the future time period may also be set by the user) based on the analysis of the pollutant concentration data and meteorological data within the predetermined time period. The neural network model may predict the pollutant concentration data in the future period (the length of the future period may also be set by the user) based on the analysis of the pollutant concentration data and meteorological data in the set period. The parameters of the neural network model may be optimized based on gradients, such as the adaptive momentum algorithm Adam, which allows the process of optimizing the model parameters to converge faster and helps to achieve the optimal solution in fewer steps. Embodiments of the present disclosure do not limit the method of optimizing the parameters Θ of the neural network model.

The model parameters Θ are further modified by using the validation data Xᵥₐₗ in the validation set and the test data Xₜₑₛₜ in the test set, i.e., the parameters Θ are adjusted according to the performance of the neural network model on the validation data set to improve the prediction accuracy of the neural network model and obtain better generalization performance.

This disclosure embodiment forecasts air pollutant concentrations based on graph neural networks and using deep learning algorithms, and by constructing the spatial distribution of air quality monitoring stations arranged in the target area as a graph-structured adjacent matrix and using graph neural networks to model the spatial variation of air pollutant concentrations in the area, it may capture the variation of air pollutant concentrations in a larger area more effectively, and, by combining with By combining the modeling analysis with pollutant concentration data and meteorological data in the target area for a predetermined length of time, the temporal and spatial dynamic characteristics of air pollutants are effectively integrated and utilized to improve the prediction accuracy and obtain a strong generalization capability.

In one exemplary embodiment, constructing a training set, a validation set, and a test set based on the data set, comprising:

Read pollutant concentration data and meteorological data from the data set, where the pollutant concentration data includes the value of each pollutant concentration and the meteorological data includes the value of various meteorological conditions;

According to the input time length tᵢₙ and output time length tₒᵤₜ required for forecasting, the data set is obtained by sliding window slicing operation in the time dimension to obtain multiple data fragments, which are all of length t in the time dimension, and the characteristic dimensions of these multiple data fragments are the values of various meteorological conditions and the values of each pollutant concentration, where t=tᵢₙ+tₒᵤₜ;

The training set, validation set and test set are constructed based on the obtained multiple data fragments.

The forecasting of air pollutant concentrations using the modified neural network model in the above step S103 may be that the neural network model analyzes the data within the input time length tᵢₙ and predicts the pollutant concentration data within the output time length tₒᵤₜ accordingly. Thus, the data in the dataset is previously divided into a plurality of data fragments, each of which has a length t=tᵢₙ+tₒᵤₜ in the time dimension, and the training set, validation set and test set are constructed based on the obtained plurality of data fragments, which may facilitate training and parameter adjustment of the neural network model.

The input time length tᵢₙ and output time length tₒᵤₜ may be set as needed, for example, tᵢₙ may be set to 72 hours and tₒᵤₜ to 24 hours, and this disclosure embodiment does not limit this.

In constructing the training set, validation set and test set, the data may be selected among a plurality of data fragments as needed, or divided in a certain ratio, e.g., all data fragments of the data set may be quantitatively divided into 70%, 10% and 20%, and assigned to the training set, validation set and test set, respectively. Embodiments of this disclosure do not limit the way in which the data is selected or divided when constructing the training set, validation set, and test set.

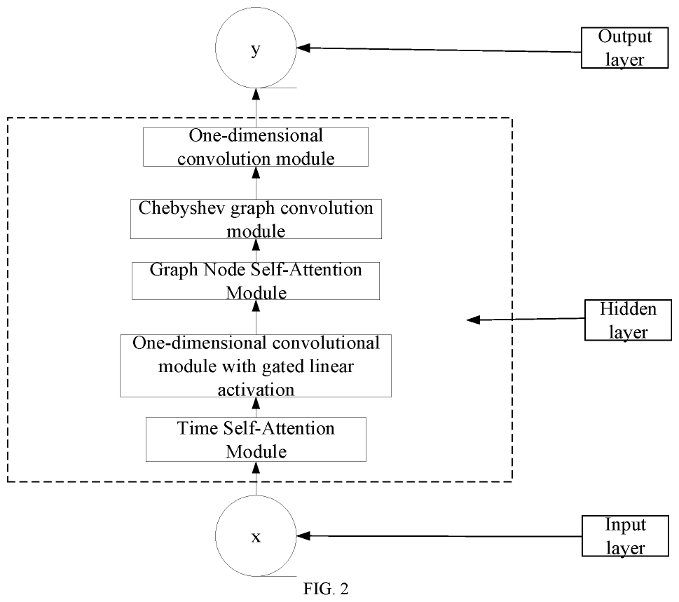

FIG. 2 is a schematic diagram of the neural network model in this disclosure embodiment, and the air pollutant concentration forecasting method of this disclosure embodiment is described below in conjunction with FIG. 2.

An exemplary embodiment in which a neural network model is trained using a training set, comprising:

The data (x, y) in the training set are fed into the neural network model F(x; Θ|A) by batch for training, and the predicted output ŷ is obtained to calculate the loss function l(ŷ, y), and the parameters Θ of the model are optimized based on the gradient descent algorithm;

where y is the pollutant concentration data; the neural network model F(x; Θ|A) includes: an input layer, a hidden layer and an output layer, and the hidden layer includes multiple self-attentive modules based on graph convolutional layers and one-dimensional convolutional layers.

The batches of training set data Xₜᵣₐᵢₙ may be divided as needed, and (x, y)∈Xₜᵣₐᵢₙ may be fed into the neural network model for training by batch, and there is no restriction on how the training set data may be divided in this disclosure embodiment.

In an exemplary embodiment, the neural network model F(x; Θ|A) may include: a linear input layer, a hidden layer and a linear output layer, and a linear input layer and a linear output layer may make a linear mapping of the input data of their respective layers, assuming that the input is x and the output is y, then the linear layer may be expressed as y=xw+b, where w and b are the linear layer parameters, and the linear layer parameters of the linear input layer and the linear output layer may be set as needed, and this disclosure embodiment does not limit this.

The hidden layer of the neural network model F(x; Θ|A) may include a stack of two sets of modules based on the self-attention mechanism, such as the temporal self-attention module and the graph node self-attention module. By introducing the modules based on the self-attention mechanism, it may effectively improve the ability of the network to extract features and utilize the data more fully, which helps to improve the efficiency and accuracy of prediction, and at the same time makes the constructed neural network model with high generalization ability, which is convenient for practical disclosure and has greater practical significance.

In an exemplary embodiment, the adjacent matrix A of the graph structure is constructed based on the spatial distribution of monitoring stations in the target area, comprising:

The longitude and latitude coordinates of the monitoring stations are read from the data set, converted to relative coordinates in the Cartesian coordinate system, and the following adjacent matrix is constructed based on the distance between every two monitoring stations:

$${A}_{ij} = \{ \begin{matrix}exp ( - \frac{{d}_{ij}^{2}}{{σ}^{2}} ) , & {d}_{ij} \geq κ , \\ 0 , & {d}_{ij} < κ ,\end{matrix}$$

where Aij is the element of the ith row and jth column of the adjacent matrix, dij denotes the distance between monitoring station i and monitoring station j, σ is the standard deviation of the distance between all monitoring stations, and κ is a preset hyperparameter for ensuring the sparsity of the adjacent matrix.

The preset hyperparameter κ (i.e., Kappa) is used to ensure that there are enough zeros in the adjacent matrix as to ensure the sparsity of the graph connectivity. In this disclosure embodiment, κ is set to 0.1, however, this value may be set according to the demand, and this disclosure embodiment does not limit this.

According to the longitude and latitude coordinates of air quality monitoring stations, the spatial distribution of monitoring stations may be obtained, and the air pollutant concentration data may be captured in a wider range, combined with the air pollutant concentration data in the target area over a period of time, that is, the spatial and temporal variation characteristics of air pollutant concentration may be modeled jointly, so that the whole model has stronger expressive ability and higher prediction accuracy.

An exemplary embodiment in which the input data x of the neural network model F(x; Θ|A) is z-score normalized in the characteristic dimension so that the input data x has zero mean and unit standard deviation, that is:

$${x}_{norm}^{( c )} = \frac{{x}^{( c )} - {μ}^{( c )}}{{σ}^{( c )}} ,$$

where the superscript (c) denotes the cth characteristic dimension, xₙₒᵣₘ^{(c)}, denotes the input data x that has been z-score normalized in the cth characteristic dimension, pt denotes the mean of the data points, and a denotes the standard deviation of the distance between all monitoring stations; after the input data x is z-score normalized in the characteristic dimension, the input data x has zero mean and unit standard deviation. At this time, the input neural network model F(x; Θ|A) is trained, which is more conducive to the neural network model for learning.

The dimension of the composition tensor of the input data x of the neural network model is N×T×G×C, where N denotes batch, T denotes time, G denotes graph node, and C denotes characteristic dimension, as follows.

In one exemplary embodiment, the data are operated in the hidden layer in the following order:

A temporal self-attention operation, followed by a one-dimensional convolution operation with gated linear activation in time dimension, a graph node self-attention operation, a first-order Chebyshev graph convolution operation with residual connectivity in graph node dimension, and a one-dimensional convolution operation with residual connectivity in time dimension;

wherein, in the above operation, the result data of the operation that comes first in order is used as the input data for the next operation.

It may be the self-attentive module in the hidden layer that performs the above operations.

In one exemplary embodiment, a temporal self-attention operation, comprising:

When the dimension of the input data x₁ of the time-self-attention operation is N×T×G×C, the input data xᵢ is operated by two graph convolution layers φ_{G} and θ_{G}, and the operations yield z₁=φ_{G}(x₁|A) and z₂=θ_{G} (x₁|A), respectively.

Reshape the features z₁,z₂ obtained after the operation to have dimension N×T×GC and do softmax operation by row after multiplying by batch matrix in the last dimension to obtain the self-attentive relation a₁=softmax(bmm(z₁^{T}, z₂)), the dimension of the self-attentive relation a₁ is N×T×T.

Then the self-attentive relation a1 is multiplied right on the linear mapping of the input data x1 by batch, and after multiplying by the predetermined scaling factor γ, it is deformed into NxTxGxC, and then added with the input data x1 to obtain the result of the temporal self-attention operation y1 and output, i.e., y1=x1+γbmm(ψ(x1), a1), where ψ is the linear layer and y1 is the output of the temporal self-attention module. The scaling factor γ is used to ensure that the change in attention is small enough to make the neural network model easier to train, and the value of the scaling factor γ may be set to 0.1, however, the present disclosure embodiment does not limit the setting of the value of the scaling factor γ.

In an exemplary embodiment, a one-dimensional convolutional operation with gated linear activation in the time dimension, comprising:

When the dimension of the input data x2 of the one-dimensional convolution operation with gated linear activation in the time dimension is N×L×C₁, the gated linear activation performs the convolution operation on the input data x₂ in dimension L to obtain the output z, the dimension of z is N×L×2C₂, where L is the length of a single sample, C₁ is the characteristic dimension of the input data x₂, and C₂ is the dimension of the set output;

Split z in half according to the characteristic dimension into z₃, z₄, whose dimensions are N×L×C₂, z₃ will be activated by the sigmoid function and multiply z₄ by elements to get the operation result y₂ and output, i.e. That is, y₂=sigmoid(z₃)⊗z₄, where ⊗ denotes element-by-element multiplication and y₂ is the output of the 1D convolution module with gated linear activation.

In one exemplary embodiment, the graph node self-attention operation, comprising:

When the dimension of the input data x₃ of the graph node self-attention operation is N×T×G×C, the input data x_3 is operated by two one-dimensional convolutional layers φ_{T} and θ_{T}, and the operations yield z₅=φ_{T}(x₃) and z₆=θ_{T}(x₃), respectively;

Reshape the features z₅, z₆ obtained after the operation to have dimension N×G×TC and do softmax operation by row after multiplying by batch matrix in the last dimension to obtain the self-attentive relation a₂=softmax (bmm(z₅^{T}, z₆)), the dimension of the self-attentive relation a₂ is N×G×G;

Then the self-attentive relation a₂ is multiplied right on the linear mapping of the input data x₃ by batch and deformed to NxTxGxC after multiplying by the predetermined scaling factor γ. Then it is added with the input data x₃ to obtain the result of the graph node self-attention operation y₃ and output, i.e., y₃=x₃+γbmm(ψ(x₃), a₂), where ψ is the linear layer.

An exemplary embodiment of a first-order Chebyshev graph convolution operation in graph node dimension with residual connectivity, comprising:

For each node, the features of itself and other nodes in the one-hop neighborhood are calculated separately and normalized symmetrically according to the degree matrix, that is

$${g}_{θ} ( {x}_{4} ) = {θ}_{0} + {θ}_{1} {x}_{4} + {θ}_{2} {D}^{- \frac{1}{2}} {AD}^{- \frac{1}{2}} {x}_{4} ,$$

where θ is a parameter, x₄ is the input data for carrying out the convolution operation of the first-order Chebyshev graph of graph node dimensions with residual connections, D is the symmetry matrix, and Dᵢᵢ=ΣⱼAᵢⱼ is the degree matrix of the graph;

After the first-order Chebyshev map convolution operation, the result y₄ is obtained and output, y₄=x₄+LReLU(g_{θ}(x₄)), LReLU is the LeakyReLU activation function, and its negative semi-axis slope may be set to 0.1, which is not limited in this disclosure. y₄ is the output of the Chebyshev map convolution module.

In one exemplary embodiment, a one-dimensional convolutional operation with residual connections in the time dimension may be performed in a manner well known to those skilled in the art, and will not be described here.

An exemplary embodiment in which the loss function is a smoothed L1 loss function

For each prediction [symbol] , calculate the value of the error

$${e}_{i} = \vert - {y}_{i} \vert {-}_{s}$$

where s is the hyperparameter controlling the size of the error, and the loss function is lᵢ=0.5eᵢ² when eᵢ<1, lᵢ=eᵢ−0.5 when eᵢ≥1, and the total loss function is

$$l = \frac{1}{N} {Σ}_{i} {l}_{i} .$$

The use of a smoothed L1 loss function may make the training process of the neural network model more stable and may accelerate convergence, or you may use a L2, oL1, etc. loss function for regression problems, and this disclosure embodiment is not limited to the choice of loss function.

Compared with traditional methods and other deep learning-based methods for forecasting air pollutant concentrations, the method of this disclosure embodiment collects air data from multiple air quality monitoring stations in the target region over a long period of time; pre-processes these data to obtain a dataset for training, validation and testing; uses the training data to train a deep learning model based on graph neural networks, iteratively calculates and optimizes the loss function, and obtains a forecast The training data is used to train a deep learning model based on graph neural network, and the loss function is iteratively computed and optimized to obtain a model with better forecasting capability. The method jointly models the spatial and temporal variability of air pollutant concentrations and introduces a self-attentive mechanism, which has stronger expressive power and effectively reduces the errors in air pollutant concentration prediction.

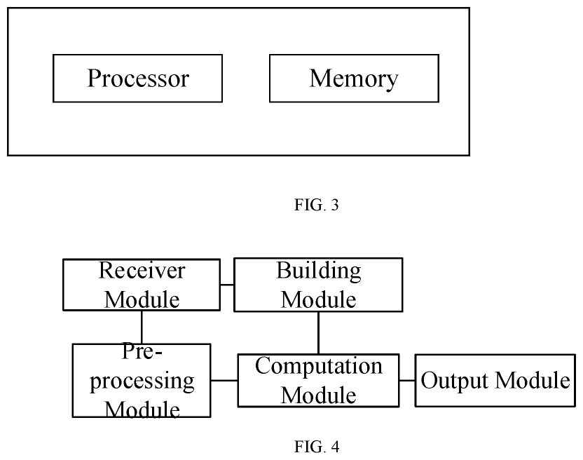

As shown in FIG. 3, embodiments of the present disclosure also provide an air pollutant concentration forecasting device comprising a memory and a processor, the memory for storing a program for performing air pollutant concentration forecasting; the processor for reading the program for performing air pollutant concentration forecasting and executing the air pollutant concentration forecasting method in any of the above embodiments.

In one exemplary embodiment, as shown in FIG. 4, the air pollutant concentration forecasting device provided in this disclosure embodiment may include a receiving module, a pre-processing module, a building module, a learning module, and an output module.

The receiving module of the air pollutant concentration forecasting device in FIG. 4 is used to receive settings from the user, such as: the selected target area, what kinds of air pollutant concentration data and meteorological data are needed, the input time length, the output time length, the temporal resolution of the data, and the data allocation ratio of the training set, the validation set and the test set, the hyperparameters to be used by the neural network model, etc. in the above embodiment need to be initially set by the user. The receiving module may provide the prepared configuration options for the user to choose (e.g., the configurable options are displayed on the panel and the user may choose, fill in or delete them), or the user may input them directly, and the user may input all the setting parameters at once or in steps according to the aforementioned method flow, and the embodiment of this disclosure does not make any There is no restriction on the way and form of receiving user settings by the receiving module.

After the receiver module receives the user's settings, for the data collected by the pre-set multiple sensors:

Pre-processing operations such as filling missing values and slicing by the pre-processing module according to the user's settings for data such as air pollutant concentration data and meteorological data, and organizing to obtain the data set, on the basis of which the training set, validation set and test set are constructed;

The adjacent matrix A of the graph structure is constructed by the construction module from the data of the spatial distribution of the monitoring stations.

The learning module builds the neural network model F(x; Θ|A) based on the data sent from the preprocessing module and the building module, as well as the user's settings, and performs the process of training and parameter adjustment of the neural network model to obtain the modified neural network model; then, according to the user's setting rules (e.g., input time length, output time length, etc.), the modified neural network model is used to forecast the air pollutant concentration, and the output module outputs the forecast results.

This air pollutant concentration forecasting device may implement the forecasting method of air pollutant concentration in any of the above embodiments, and the details of implementation are not repeated here.

Embodiments of the present disclosure also provide a computer-readable storage medium storing computer-executable instructions, the described computer-executable instructions being used to perform the air pollutant concentration forecasting method of any of the above embodiments.

The method for forecasting air pollutant concentrations in an embodiment of this disclosure is illustrated below by Example 1.

### Example 1

First, a target city is selected and air pollutant concentration data, including: PM10, PM2.5, O3, SO2, NOx, etc., and meteorological data, including: temperature, humidity, wind speed, wind direction, atmospheric pressure, etc., are collected from the target city at 1-hour intervals from 0:00 on Sep. 1, 2017 to 23:00 on Mar. 31, 2018; the first 20 and last 20 data points with missing data are used The linear average of data points to fill in the time missing data points of the value to fill the missing values, organized into data sets.

Then, according to the input time length tᵢₙ=72 h and output time length tₒᵤₜ=24 h needed to carry out the forecast, the data set is sliding-window slicing operation is performed in the time dimension so that the length of each data segment t=tᵢₙ+tₒᵤₜ=96 h, and based on this, the training set, validation set and test set are constructed with the proportions of 70%, 10% and 20%, respectively.

In addition, the adjacent matrix A of the graph structure is constructed based on the spatial distribution of the 35 monitoring stations in the target city by converting the longitude and latitude coordinates of each monitoring station into relative coordinates in the Cartesian coordinate system, calculating the two-by-two distance between stations, and constructing the following adjacent matrix:

$${A}_{ij} = \{ \begin{matrix}exp ( - \frac{{d}_{ij}^{2}}{{σ}^{2}} ) , & {d}_{ij} \geq κ , \\ 0 , & {d}_{ij} < κ ,\end{matrix}$$

where dᵢⱼ denotes the distance between monitoring station i and monitoring station j, a is the standard deviation of all distances, κ=0.1 is the hyperparameter that guarantees the sparsity of the adjacent matrix, and Aᵢⱼ is the element of the i-th row and j-th column of the adjacent matrix.

Then, a neural network model F(x; Θ|A) is built, which consists of an input linear layer, two self-attentive modules and an output linear layer with an internal characteristic dimension of 128. The self-attentive module performs first a temporal self-attention operation on the input data, then a one-dimensional convolution operation with gated linear activation in time dimension, a graph node self-attention operation, a graph first-order Chebyshev graph convolution operation with residual connectivity in node dimension, and one one-dimensional convolution operation with residual connectivity in time dimension.

For the temporal self-attentive module: the input data x₁ has dimension N×T×G×C, which will be calculated by two graph convolution layers to get z₁=φ_{G}(x₁|A) and z₂=θ_{G}(x₁|A), where φ_{G} and θ_{G} denote the graph convolution layer; the transformed features z₁, z₂ are deformed to dimension N×T×GC, and in the last dimension by batch matrix After multiplication, do softmax operation by row, and get the self-attentive relation a₁=softmax(bmm(z₁^{T}, z₂)) with dimension N×T×T; then multiply this relation by batch right on the linear map of input data x₁ and multiply by scaling factor γ=0.1, and finally deform to N×T×G×C, and input data x₁ is added, i.e., y₁=x₁+γbmm(ψ(x₁), a), where ψ is the linear layer, to obtain the module output.

For the one-dimensional convolution module with gated linear activation: the dimension of the input data x₂ is N×L×Cᵢ, where L is the length of a single sample and C₁ is the characteristic dimension of the input data. The gated linear activation performs the convolution operation on the input data x₂ in dimension L to obtain the output z dimension as N×L×2C₂, where C₂ is the dimension of the output of the set module. Split z in half according to the characteristic dimension into z₃, z₄, and the dimensions of z₃, z₄ are both N×L×C₂. The output of the module y₂=sigmoid(z₃)⊗z₄ is obtained by multiplying z₃ by element after activation of the sigmoid function, where ⊗ denotes element-by-element multiplication.

For the self-attentive module for graph nodes: the dimension of input data x₃ is N×T×G×C, and it will be calculated by two one-dimensional convolution layers to get z₅=φ_{T}(x₃) and z₆=θ_{T}(x₃), where φ_{T} and θ_{T} denote one-dimensional convolution layers; the transformed features z₅, z₆ are deformed to dimension N×G×TC and multiplied by batch matrix in the last dimension After that, do the softmax operation by row to get the self-attentive relation a₂=softmax(bmm(z₁^{T}, z₂)) with dimension N×G×G; then multiply this relation by batch right on the linear mapping of the input data and multiply by the scaling factor γ=0.1, and finally deform to N×T×G×C, and add with the input data x₃, that is, y₃=x₃+γbmm(ψ(x₃), a₂), where w is the linear layer, to obtain the module output y₃.

For the Chebyshev graph convolution module: for each node, the features of itself and other nodes in the one-hop neighborhood are calculated and normalized symmetrically according to the degree matrix, i.e.,

$${g}_{θ} ( {x}_{4} ) = {θ}_{0} + {θ}_{1} {x}_{4} + {θ}_{2} {D}^{- \frac{1}{2}} {AD}^{- \frac{1}{2}} {x}_{4} , ,$$

where θ is the parameter, D is the symmetry matrix, Dᵢᵢ=ΣⱼAᵢⱼ is the graph degree matrix, and the output is y₄=x₄+LReLU(g_{θ}(x₄)), LReLU is the LeakyReLU activation function, and the slope of its negative half-axis is taken as 0.1 here.

The training set data (x, y)∈Xₜᵣₐᵢₙ is fed into the neural network model for training by batch, and the dimension of the input data x is N (batch)×T (time)×G (graph node)×C (characteristic dimension), and z-score normalization is done on the characteristic dimension before input to the neural network model so that it has zero mean and unit standard deviation, that is

$${x}_{norm}^{( c )} = \frac{{x}^{( c )} - {μ}^{( c )}}{{σ}^{( c )}} ,$$

where the superscript (c) denotes the cth characteristic dimension, μ denotes the mean of the data points, and a denotes the standard deviation of the data points. After the input is calculated as described above, the predicted output ŷ is obtained, the loss function [symbol] (ŷ,y) is calculated, and the parameter Θ of the gradient optimization model is based.

In training, the loss function used is a smooth one-parameter loss function, and for each prediction [symbol] calculates eᵢ=| [symbol] −yᵢ|/s, where s is the hyperparameter controlling the size of the error, and the loss function [symbol] =0.5eᵢ² when eᵢ<1, the loss function is [symbol] ᵢ=eᵢ−0.5 when eᵢ≥1, and the total loss function is

$$ℓ = \frac{1}{N} {Σ}_{i} {ℓ}_{i} .$$

1. A method for forecasting air pollutant concentrations, wherein the method comprises:
constructing a training set, a validation set, and a test set based on a data set; wherein the data set is obtained by collecting pollutant concentration data and meteorological data in a target area for a predetermined length of time;
constructing an adjacent matrix A of a graph structure based on a spatial distribution of monitoring stations in the target area;
building a neural network model F(x;Θ|A), wherein x is input data of the neural network model, the input data comprises the pollutant concentration data and the meteorological data during a set time period,
training the neural network model by using data of the training set,
adjusting parameters Θ of the neural network model by using data of the validation set and data of the test set to obtain a modified neural network model;
forecasting the air pollutant concentrations, by using the modified neural network model.

2. The method for forecasting the air pollutant concentrations according to claim 1, wherein the step of constructing the training set, the validation set, and the test set based on the data set, comprises:
reading the pollutant concentration data and the meteorological data from the data set, wherein the pollutant concentration data comprises values for individual pollutant concentrations and the meteorological data comprises values for various meteorological conditions;
according to an input time length tᵢₙ and an output time length tₒᵤₜ needed for forecasting, obtaining a plurality of data segments after sliding window slicing operation of the data set in a time dimension, wherein lengths of the plurality of data segments in the time dimension are t, characteristic dimensions of the plurality of data segments are the values for the various meteorological conditions and the values for the individual pollutant concentrations, wherein t=tᵢₙ+tₒᵤₜ;
constructing the training set, the validation set, and the test set based on the plurality of data segments.

3. The method for forecasting the air pollutant concentrations according to claim 1, wherein the step of training the neural network model by using the data of the training set, comprises:
feeding data (x,y) in the training set into the neural network model in a batch for training to obtain a predicted output ŷ,
calculating a loss function [symbol] (ŷ, y), and
optimizing the parameters Θ of the model based on a gradient descent algorithm;
wherein y is the pollutant concentration data; the neural network model comprises: an input layer, a hidden layer, and an output layer, the hidden layer comprises a plurality of self-attentive modules based on a graph convolution layer and a one-dimensional convolution layer.

4. The method for forecasting the air pollutant concentrations according to claim 1, wherein the step of constructing the adjacent matrix A of the graph structure based on the spatial distribution of the monitoring stations in the target area, comprises:
reading latitude and longitude coordinates of the monitoring stations from the data set, converting the latitude and longitude coordinates of the monitoring stations to relative coordinates in a Cartesian coordinate system, and constructing the adjacent matrix A based on a distance between every two of the monitoring stations:
$${A}_{ij} = \{ \begin{matrix}exp ( - \frac{{d}_{ij}^{2}}{{σ}^{2}} ) , & {d}_{ij} \geq κ , \\ 0 , & {d}_{ij} < κ ,\end{matrix}$$
where Aᵢⱼ is an element of the i-th row and j-th column of the adjacent matrix A, dᵢⱼ denotes a distance between a monitoring station i and a monitoring station j, σ is a standard deviation of a distance between all of the monitoring stations, and κ is a preset hyperparameter for ensuring a sparsity of the adjacent matrix A.

5. The method for forecasting the air pollutant concentrations according to claim 1, wherein the input data x of the neural network model is z-score normalized in a characteristic dimension to obtain z-score normalized input data x, the z-score normalized input data x has a zero mean and a unit standard deviation:
$${x}_{norm}^{( c )} = \frac{{x}^{( c )} - {μ}^{( c )}}{{σ}^{( c )}} ,$$
where superscript (c) denotes a cth characteristic dimension, xₙₒᵣₘ^{(c)} denotes the z-score normalized input data x on the cth characteristic dimension, μ denotes a mean of data points, and σ denotes a standard deviation of a distance between all of the monitoring stations;
a dimension of a composition tensor of the input data x of the neural network model is N×T×G×C;
where N denotes a batch size, T denotes time, G denotes a graph node, and C denotes the characteristic dimension.

6. The method for forecasting the air pollutant concentrations according to claim 3, wherein the data (x,y) are computed in the hidden layer in the following order:
a temporal self-attention operation, followed by a one-dimensional convolution operation with a gated linear activation in a time dimension, a graph node self-attention operation, a first-order Chebyshev graph convolution operation with a residual connectivity in a graph node dimension, and a one-dimensional convolution operation with the residual connectivity in the time dimension;
wherein, result data of an operation, coming first in order, is configured as input data for a next operation.

7. The method for forecasting the air pollutant concentrations according to claim 6, wherein the temporal self-attention operation comprises:
when a dimension of first input data x₁ of the temporal self-attention operation is N×T×G×C, operating the first input data x₁ by two graph convolution layers φ_{G} and θ_{G} respectively, to obtain z₁=φ_{G}(x₁|A) and z₂=θ_{G} (x₁|A) respectively;
reshaping z₁, z₂ in a dimension N×T×GC and doing a softmax operation by a row after multiplying by a batching matrix in the last dimension to obtain a self-attentive relation a₁=softmax(bmm(z₁^{T}, z₂)), wherein the self-attentive relation a₁ is in a dimension N×T×T;
multiplying the self-attentive relation a₁ right on a linear mapping of the first input data x₁ by a batching and multiplying the self-attentive relation a₁ by a predetermined scaling factor γ, to deform the self-attentive relation a₁ into N×T×G×C, and then adding the self-attentive relation a₁ with the first input data x₁ to obtain a result of the temporal self-attention operation y₁ and outputting the result.

8. The method for forecasting the air pollutant concentrations according to claim 6, wherein the one-dimensional convolution operation with the gated linear activation in the time dimension comprises:
when a dimension of second input data x₂ of the one-dimensional convolution operation with the gated linear activation in the time dimension is N×L×Cᵢ, activating a result of an one-dimensional convolution operation on the second input data x₂ in a dimension L to obtain an output z having a dimension of N×L×2C₂, where L is a length of a single sample, C₁ is a characteristic dimension of the second input data x₂, and C₂ is a dimension of a set output;
splitting the output z into z₃, z₄ according to the characteristic dimension in half, wherein dimensions of z₃, z₄ are both N×L×C₂; a result of multiplying z₃ by elements by z₄ after activating z₃ by a sigmoid function to obtain an operation y₂, and outputting y₂=sigmoid(z₃)⊗z₄, where ⊗ denotes an element-wise multiplication.

9. The method for forecasting the air pollutant concentrations according to claim 6, wherein the graph node self-attention operation, comprising:
when a dimension of third input data x₃ of the graph node self-attention operation is N×T×G×C, operating the third input data x₃ by two one-dimensional convolution layers φ_{T} and θ_{T} respectively, to obtain z₅=φ_{T}(x₃) and z₆=θ_{T}(x₃) respectively;
reshaping z₅, z₆ in a dimension N×G×TC and doing a softmax operation by row after multiplication by a batching matrix in the last dimension to obtain a self-attentive relation a₂=softmax(bmm(z₅^{T}, z₆)), wherein a dimension of the self-attentive relation a₂ is N×G×G;
multiplying the self-attentive relation a₂ right on a linear mapping of the third input data x₃ by batching and reshaping the self-attentive relation a₂ to N×T×G×C after multiplying by a predetermined scaling factor γ; summing the self-attentive relation a₂ with the third input data x₃ to obtain a result of the graph node self-attention operation y₃ and outputting the result.

10. The method for forecasting the air pollutant concentrations according to claim 6, wherein the first-order Chebyshev graph convolution operation with the residual connection in the graph node dimension comprises:
for each node, calculating separately and normalizing symmetrically features of each node and other nodes in a one-hop neighborhood according to a degree matrix:
$${g}_{θ} ( {x}_{4} ) = {θ}_{0} + {θ}_{1} {x}_{4} + {θ}_{2} {D}^{- \frac{1}{2}} {AD}^{- \frac{1}{2}} {x}_{4} ,$$
where θ is a parameter, x₄ is fourth input data for carrying out the first-order Chebyshev graph convolution operation with the residual connection in the graph node dimension, D is a symmetry matrix, Dᵢᵢ=ΣⱼAᵢⱼ is a degree matrix of a graph;
after performing the first-order Chebyshev graph convolution operation, obtaining and outputting a result y₄, wherein y₄=x₄+LReLU(g_{θ}(x₄)), and LReLU is a LeakyReLU activation function.

11. The method for forecasting the air pollutant concentrations according to claim 3, wherein the loss function is a smoothed L1 loss function,
for each predicted output ŷᵢ calculating a value of an error eᵢ, eᵢ=|ŷᵢ−yᵢ|/s,
where s is a hyperparameter controlling a size of the error, and the loss function is lᵢ=0.5eᵢ² when eᵢ<1, [symbol] ᵢ=eᵢ−0.5 when eᵢ≥1, and a total loss function is
$$ℓ = \frac{1}{N} {Σ}_{i} {ℓ}_{i} .$$

12. An air pollutant concentration forecasting device, comprising a memory and a processor, wherein the memory is configured to store a program for performing an air pollutant concentration forecasting; the processor is configured to read the program for performing the air pollutant concentration forecasting and to execute the method for forecasting the air pollutant concentrations as claimed in claim 1.

13. A computer-readable storage medium storing computer-executable instructions, wherein the computer-executable instructions are configured to perform the method of claim 1.

14. The method for forecasting the air pollutant concentrations according to claim 2, wherein the input data x of the neural network model is z-score normalized in a characteristic dimension to obtain z-score normalized input data x, the z-score normalized input data x has a zero mean and a unit standard deviation:
$${x}_{n o r m}^{( c )} = \frac{{x}^{( c )} - {μ}^{( c )}}{{σ}^{( c )}} , \qquad ( c )$$
where superscript (c) denotes a cth characteristic dimension, xₙₒᵣₘ^{(c)} denotes the z-score normalized input data x on the cth characteristic dimension, μ denotes a mean of data points, and σ denotes a standard deviation of a distance between all of the monitoring stations;
a dimension of a composition tensor of the input data x of the neural network model is N×T×G×C;
where N denotes a batch size, T denotes time, G denotes a graph node, and C denotes the characteristic dimension.

15. The method for forecasting the air pollutant concentrations according to claim 3, wherein the input data x of the neural network model is z-score normalized in a characteristic dimension to obtain z-score normalized input data x, the z-score normalized input data x has a zero mean and a unit standard deviation:
$${x}_{norm}^{( c )} = \frac{{x}^{( c )} - {μ}^{( c )}}{{σ}^{( c )}} ,$$
where superscript (c) denotes a cth characteristic dimension, xₙₒᵣₘ^{(c)} denotes the z-score normalized input data x on the cth characteristic dimension, μ denotes a mean of data points, and σ denotes a standard deviation of a distance between all of the monitoring stations;
a dimension of a composition tensor of the input data x of the neural network model is N×T×G×C;
where N denotes a batch size, T denotes time, G denotes a graph node, and C denotes the characteristic dimension.

16. The method for forecasting the air pollutant concentrations according to claim 4, wherein the input data x of the neural network model is z-score normalized in a characteristic dimension to obtain z-score normalized input data x, the z-score normalized input data x has a zero mean and a unit standard deviation:
$${x}_{norm}^{( c )} = \frac{{x}^{( c )} - {μ}^{( c )}}{{σ}^{( c )}} ,$$
where superscript (c) denotes a cth characteristic dimension, xₙₒᵣₘ^{(c)} denotes the z-score normalized input data x on the cth characteristic dimension, μ denotes a mean of data points, and σ denotes the standard deviation of the distance between all of the monitoring stations;
a dimension of a composition tensor of the input data x of the neural network model is N×T×G×C;
where N denotes a batch size, T denotes time, G denotes a graph node, and C denotes the characteristic dimension.

17. The air pollutant concentration forecasting device according to claim 12, wherein the step of constructing the training set, the validation set, and the test set based on the data set, comprises:
reading the pollutant concentration data and the meteorological data from the data set, wherein the pollutant concentration data comprises values for individual pollutant concentrations and the meteorological data comprises values for various meteorological conditions;
according to an input time length tᵢₙ and an output time length tₒᵤₜ needed for forecasting, obtaining a plurality of data segments after sliding window slicing operation of the data set in a time dimension, wherein lengths of the plurality of data segments in the time dimension are t, characteristic dimensions of the plurality of data segments are the values for the various meteorological conditions and the values for the individual pollutant concentrations, wherein t=tᵢₙ±tₒᵤₜ;
constructing the training set, the validation set, and the test set based on the plurality of data segments.

18. The air pollutant concentration forecasting device according to claim 12, wherein the step of training the neural network model by using the data of the training set, comprises:
feeding data (x,y) in the training set into the neural network model in a batch for training to obtain a predicted output ŷ,
calculating a loss function [symbol] (ŷ,y), and
optimizing the parameters Θ of the model based on a gradient descent algorithm;
wherein y is the pollutant concentration data; the neural network model comprises: an input layer, a hidden layer, and an output layer, the hidden layer comprises a plurality of self-attentive modules based on a graph convolution layer and a one-dimensional convolution layer.

19. The air pollutant concentration forecasting device according to claim 12, wherein the step of constructing the adjacent matrix A of the graph structure based on the spatial distribution of the monitoring stations in the target area, comprises:
reading latitude and longitude coordinates of the monitoring stations from the data set, converting the latitude and longitude coordinates of the monitoring stations to relative coordinates in a Cartesian coordinate system, and constructing the adjacent matrix A based on a distance between every two of the monitoring stations:
$${A}_{ij} = \{ \begin{matrix}exp ( - \frac{{d}_{ij}^{2}}{{σ}^{2}} ) , & {d}_{ij} \geq κ , \\ 0 , & {d}_{ij} < κ ,\end{matrix}$$
where Aᵢⱼ is an element of the i-th row and j-th column of the adjacent matrix A, dᵢⱼ denotes a distance between a monitoring station i and a monitoring station j, σ is a standard deviation of a distance between all of the monitoring stations, and κ is a preset hyperparameter for ensuring a sparsity of the adjacent matrix A.

20. The air pollutant concentration forecasting device according to claim 12, wherein the input data x of the neural network model is z-score normalized in a characteristic dimension to obtain z-score normalized input data x, the z-score normalized input data x has a zero mean and a unit standard deviation:
$${x}_{norm}^{( c )} = \frac{{x}^{( c )} - {μ}^{( c )}}{{σ}^{( c )}} ,$$
where superscript (c) denotes a cth characteristic dimension, xₙₒᵣₘ^{(c)} denotes the z-score normalized input data x on the cth characteristic dimension, μ denotes a mean of data points, and σ denotes a standard deviation of a distance between all of the monitoring stations;
a dimension of a composition tensor of the input data x of the neural network model is N×T×G×C;
where N denotes a batch size, T denotes time, G denotes a graph node, and C denotes the characteristic dimension.
